(19) United States
(12) Patent Application Publication
(10) Pub. No.: US 2005/0244810 A1
(43) Pub. Date: Nov. 3, 2005

(54) Taste signaling in gastrointestinal cells

(76) Inventors: Josephine M. Egan, Baltimore, MD (US); Stephen A. Gravine, Rutherford, NJ (US); Robert F. Margolskee, Upper Montclair, NJ (US); Richard A. McGregor, Rutherford, NJ (US); Lenore A. Snyder, New York, NY (US); Michael Theodorakis, Baltimore, MD (US)

Correspondence Address: Michael L. Goldman;Nixon Peabody LLP, Clinton Square, P.O. Box 31051, Rochester, NY, 14603-1051, US

(21) Appl. No.: 10/949,751

(22) Filed: Sep. 24, 2004

Related U.S. Application Data
(60) Provisional application No. 60/507,204, filed on Sep. 29, 2003.

(51) Int. Cl. C12Q 1/00

(52) U.S. Cl. 435/4

(57) ABSTRACT

Disclosed are materials and methods relevant to taste transduction. Also disclosed are human gastrointestinal cells that comprise or are capable expressing endogenous taste signaling proteins. Also disclosed are human gastrointestinal cells that comprise or are capable of expressing endogenous taste signaling proteins as well as hormones, neurotransmitters or soluble mediators of the gastrointestinal tract that are involved in or affect metabolism, digestion and appetite. Also disclosed are the uses of these human cells or their membranes to study how compounds affect taste transduction and/or metabolism, digestion and appetite, including effects on satiety, emesis and diabetes.

This application claims the benefit of U.S. Provisional Patent Application Ser. No. 60/507,204, filed Sep. 29, 2003, which is hereby incorporated by reference in its entirety.

### FIELD OF THE INVENTION

This invention generally relates to materials and methods relevant to taste transduction. More particularly, this invention relates to human gastrointestinal cells that comprise or are capable of expressing endogenous taste signaling proteins. Even more particularly, this invention relates to human gastrointestinal cells that comprise or are capable of expressing endogenous taste signaling proteins as well as hormones, neurotransmitters or soluble mediators of the gastrointestinal tract that are involved in or affect metabolism, digestion and appetite. This invention further relates to the use of these human cells or their membranes to study how compounds affect taste transduction and/or metabolism, digestion and appetite, including effects on satiety, emesis and diabetes.

### BACKGROUND OF THE INVENTION

Taste Transduction

Vertebrate taste transduction is mediated by specialized epithelial cells, referred to as taste receptor cells. These cells are organized into groups of 40-100 cells that form taste buds. Taste buds are ovoid structures, the vast majority of which are embedded within the epithelium of the tongue.

Taste transduction is initiated at the apical portion of a taste bud at the taste pore. Here the microvilli of the taste receptor cells make contact with the outside environment. Various taste stimulants (tastants) cause either depolarization (i.e., a reduction in membrane potential) or hyperpolarization (i.e., an increase in membrane potential) of taste cells and regulate neurotransmitter release from the cells at chemical synapses with afferent nerve fibers. The primary gustatory sensory fibers that receive the chemical signals enter the base of each taste bud. Lateral connections between taste cells in the same bud may also modulate the signals transmitted to the afferent nerve fibers.

The sense of taste can be divided into five primary sensations: bitter, salty, sour, sweet and umami (i.e., the response to salts of glutamic acid). Different taste modalities appear to function by different mechanisms.

Salty taste appears to be mediated by sodium ion flux through apical sodium channels [see Heck et al., Science, 223, 403-5 (1984); Schiffman et al., Proc. Natl. Acad. Sci. USA, 80, 6136-40 (1983)]. In laboratory animals, and perhaps in humans, the hormone aldosterone increases the number of these salt receptors.

Sour taste seems to be mediated via hydrogen ion blockade of potassium or sodium channels [see Kinnamon et al., J. Gen. Physiol., 91, 351-71 (1988); Kinnamon et al., Proc. Natl. Acad. Sci. USA, 85, 7023-27 (1988)].

Umami taste seems to be mediated by modified versions of metabotropic glutamate receptors known as mGluR4 [Chaudhari and Roper, Ann. N Y Acad. Sci., 855, 398-406 (1998)] and by G-protein-coupled receptors at the cell surface. Each receptor contains two subunits, designated T1R1 and T1R3, and is coupled to G proteins [see, e.g., Birnbaumer, Ann. Rev. Pharmacol. Toxicol., 30, 675-705 (1990); Simon et al., Science, 252, 802-8 (1991).

Sweet taste seems to be mediated via G-protein-coupled T1R receptors that are heterodimers of subunits T1R2 and T1R3. Bitter taste seems to be mediated by one or more G-protein coupled T2R receptors.

Briefly, G proteins are heterotrimeric proteins (each having an α, β and γ subunit) that mediate signal transduction in olfactory, visual, hormonal and neurotransmitter systems. G proteins couple cell-surface receptors to intracellular effector enzymes (e.g., phosphodiesterases and adenylate cyclase) and thereby transduce an extracellular signal into an intracellular second messenger (e.g., CAMP, cGMP, IP₃, DAG (diacylglycerol)). The α subunit of a G protein confers most of the specificity of interaction between its receptor and its effectors in the signal transduction process, while the β and γ subunits appear to be shared among different G proteins.

Some G proteins are ubiquitously expressed (e.g., Gₛ and Gᵢ), but others that are known to be involved in sensory transduction have been found only in specialized sensory cells. For example, the transducins (the G protein of the visual system—Gₜ) transduce photoexcitation in retinal rod and cone cells [see Lerea et al., Science, 224, 77-80 (1986)], and G_{olf} transduces olfactory stimulation in neurons of the olfactory epithelium [see Jones et al., Science, 244, 790-95 (1989)]. The ubiquitously expressed G proteins may also be involved in sensory transduction.

Gustducin is a taste-selective G protein [McLaughlin et al., Nature, 357, 563-69 (1992)]. Activation of gustducin triggers a cascade of intracellular reactions: activation of phosphodiesterase; degradation of 3′,5′-cyclic adenosine monophosphate (cAMP) and 3′,5′-cyclic guanosine monophosphate (cGMP); and the closing of cyclic nucleotide gated cation channels that leads to depolarization of the cell. Gustducin is homologous (˜80% identical/˜90% similar) to transducin.

In the retina, light activates rhodopsin, a G-protein-coupled receptor family, resulting in a conformational change and activation of transducin. Transducin subsequently disinhibits a cGMP-specific phosphodiesterase. The resultant decreased cGMP concentration leads to modulation of ion channel permeability, causing rod cell hyperpolarization [Birnbaumer et al., Biochem. Biophys. Acta., 1031, 163-224 (1990)].

Because of gustducin's high degree of similarity to transducin, it is thought that gustducin may be involved in taste signal transduction by modulation of a taste-specific phosphodiesterase (PDE). This hypothesis is further supported by the fact that transducin's PDE-activating domain is 86% identical and 95% similar to gustducin's, while other G proteins have much lower relatedness in this region [Rarick et al., Science, 256, 1031-33 (1992)]. Furthermore, recombinant gustducin expressed in SF9 cells has been shown to be activated by rhodopsin and can activate retinal and taste cGMP PDE [Hoon et al., Biochem. J., 309, 629-36 (1995); Ruiz-Avila et al., Nature, 376, 80-85 (1995)]. Thus, gustducin and transducin appear to be interchangeable in this regard.

Transducin has also been immunocytochemically localized to taste buds, and has been implicated in taste signal transduction by activation of a taste-specific PDE activity [Ruiz-Avila et al., Nature, 376, 80-85 (1995)]. This study and subsequent work [Ming et al., Proc. Natl. Acad. Sci. USA, 95, 8933-8 (1998)] demonstrate that taste-bud-containing membranes from bovine circumvallate papillae activate exogenously added transducin in response to bitter stimuli including denatonium, quinine, strychnine, atropine and naringen.

Gustducin has been implicated in vivo in transducing responses to bitter and sweet compounds [Wong et al., Nature, 381, 796-800 (1996)]. Gene replacement was used to generate a null mutation of the α-gustducin gene in mice. The α-gustducin knockout mice were shown to be deficient in responses to both bitter and sweet compounds as measured by two bottle preference tests as well as electrophysiology. The gustducin knockout had no effect on responses to sour or salty compounds.

Recently, putative human and rodent taste receptors for bitter taste (the T2Rs) have been cloned. Cells expressing certain of these clones respond to the bitter compounds denatonium, cyclohexamide and 6-n-propyl-2-thiouracil (PROP) [Hoon et al., Biochem. J., 309, 629-36 (1995); Adler et al., Cell, 100, 693-702 (2000); Chandrashekar et al., Cell, 100, 703-11 (2000); Matsunami et al., Nature, 404, 601-4 (2000)]. The T2R receptors appear to be specifically expressed in only the α-gustducin-positive taste receptor cells, consistent with their proposed role in bitter transduction. Also, it seems that T2Rs may link the recognition of a specific chemical structure to the perception of bitter taste [Bufe et al., Nature Genetics, 32, 3.97-401 (2002)].

Although gustducin- and transducin-mediated pathways appear to be the primary mechanism by which responses to bitter compounds are transduced, alternative mechanisms have also been proposed. Evidence thus far suggests that bitter taste transduction may be mediated by at least three mechanisms. First, as discussed above, G-protein-coupled receptors can act via gustducin/transducin [Ruiz-Avila et al., Nature, 376, 80-85 (1995); Ming et al., Proc. Natl. Acad. Sci. USA, 95, 8933-38 (1998)]. Our work and that of others suggest that at least 50% of bitter compounds couple through a receptor-dependent gustducin/transducin pathway. Second, G-protein-coupled receptors may act via G_{q} or βγ subunits to generate inositol triphosphate [Spielman et al., Physiol. Behav., 56, 1149-55 (1994); Huang et al., Nat. Neurosci., 2, 1055-62 (1999)]. A recently identified G-protein γ subunit expressed in gustducin-positive taste cells has been shown to mediate the response of certain bitter compounds to a phospholipase C (PLC)-catalyzed increase in inositol triphosphate (IP₃) [Huang et al., Nat. Neurosci., 2, 1055-62 (1999)]. This γ subunit is associated with gustducin in the taste cell. Other tastants appear to link via a different G protein (G_{q}) to IP₃ production [Spielman et al., Physiol. Behav., 56, 1149-55 (1994)]. Third, bitter-tasting molecules may act directly on G proteins and effectors such as phosphodiesterase and ion channels [Naim et al., Biochem. J., 297, 451-54 (1994); Amer and Kreighbaum, J. Pharm. Sci., 64, 1-35 (1975); Tsunenari, J. Physiol., 519, Pt 2, 397-404 (1999)].

Traditionally, sweeteners and flavorants have been used to mask the bitter taste of pharmaceuticals. The sweetener or flavorant is known to activate other taste pathways, and at sufficiently high concentration this serves to mask the bitter taste of the pharmaceutical. However, this approach has proved ineffective at masking the taste of very bitter compounds. Microencapsulation in a cellulose derivative has also been used to mask the bitter taste of pharmaceuticals; however, this approach prevents rapid oral absorption of the pharmaceutical. The nucleotide monophosphates IMP (inosine monophosphate) and GMP. (guanine monophosphate) have been used to counter the metallic or pseudo-bitter taste of KCl for its use in low-sodium edible salt compositions [Zolotov et al., U.S. Pat. No. 5,853,792]. In addition, AMP (adenosine monophosphate) has been described as an inhibitor of bitter taste [Ming et al., Proc. Natl. Acad. Sci. USA, 96, 9903-8 (1998); McGregor and Gravina, 23rd Meeting of the Association of Chemoreception Sciences (2001)].

Over the past decade substantial efforts have been directed to the development of various agents that interact with taste receptors to mimic or block natural taste stimulants [see Robert H. Cagan, Ed., Neural Mechanisms in Taste, Chapter 4, CRC Press, Inc., Boca Raton, Fla. (1989)]. Examples of agents that have been developed to mimic sweet tastes are saccharin (an anhydride of o-sulfimide benzoic acid), monellin (a protein) and the thaumatins (also proteins). Thaumatins have been used as additives in food, cigarette tips, medicines and toothpaste [Higginbotham et al, The Quality of Foods and Beverages, Academic Press, 91-111 (1981)]. However, many taste-mimicking or taste-blocking agents developed to date are not suitable as food additives because they are costly, high in calories or carcinogenic. Development of new agents that mimic or block the four basic tastes has been limited by a lack of knowledge of the taste cell proteins responsible for transducing the taste modalities. There continues to exist a need in the art for new products and methods that are involved in or affect taste detection and/or transduction. Finding human-model experimental systems to study taste detection and transduction would aid in our understanding of the molecular biology and biochemistry of taste. Such a model system would be useful for screening for novel sweeteners, enhancers of desirable flavors, or blockers of undesirable flavors.

Signal Transduction of Hormones, Neurotransmitters or Soluble Mediators in the Gastrointestinal Tract

Substantial efforts are being devoted to the development of treatments for a variety of metabolic disorders, such as body weight disorders and diabetes. Obesity is the most common nutritional disorder in the over-nourished populations of the world. Numerous studies indicate that lowering body weight dramatically reduces the risk for chronic diseases, such as diabetes, hypertension, hyperlipidemia, coronary heart disease and musculoskeletal diseases. For example, various measures of obesity, including simple body weight, waist-to-hip ratios, and mesenteric fat depot, are strongly correlated with the risk for non-insulin dependent diabetes, also known as type II diabetes. Weight reduction is a specific goal of medical treatment of many chronic diseases, including type II diabetes.

Other body weight disorders, such as anorexia nervosa and bulimia nervosa, which together affect approximately 0.2% of the female population of the western world, also pose serious health threats. Such disorders as anorexia and cachexia (wasting) are also prominent features of other diseases such as cancer, cystic fibrosis and AIDS [Tartaglia et al., U.S. Pat. No. 6,548,269].

Emesis, or nausea and vomiting, is also metabolic disorder. Common causes for emesis include medications, viral infections, seasickness or motion sickness, migraine headaches, morning sickness during pregnancy, food poisoning, food allergies, chemotherapy in cancer patients, bulimia and alcoholism.

Nausea and vomiting is also a side effect of cancer chemotherapy treatment. Approximately 70% to 80% of patients receiving cytotoxic drugs experience some degree of nausea and vomiting. Chemotherapy drugs cause nausea and vomiting because they both irritate the lining of the stomach and duodenum and stimulate nerves that lead to the vomiting center in the brain. Vomiting can be acute, occurring within minutes to hours after chemotherapy, or delayed, developing or continuing for 24 hours after chemotherapy and sometimes lasting for days. Anticipatory emesis is a conditioned or learned aversion to chemotherapy experienced by approximately 10% to 44% of cancer patients. A patient with anticipatory emesis may start vomiting before chemotherapy. Delayed emesis persists for 1 to 4 days after chemotherapy. Protracted nausea and vomiting can severely affect the patient's food intake and nutritional status.

Diabetes is a metabolic disorder that adversely affects the way the body uses sugars and starches which, during digestion, are converted into glucose. Insulin produced by the pancreas makes the glucose available to the body's cells for energy. The net effect of insulin is to promote the storage and use of carbohydrates, protein and fat. Insulin deficiency is a common and serious pathologic condition in humans [see, e.g., Altshuler et al., U.S. Pat. No. 6,562,574].

In Type I diabetes the pancreas produces little or no insulin, and insulin must be injected daily for the survival of the diabetic. In Type II diabetes the pancreas produces insulin, but the amount of insulin is insufficient and/or less than fully effective due to cellular resistance. Widespread abnormalities are associated with either form, but the fundamental defects to which the abnormalities can be traced are (1) a reduced entry of glucose into various “peripheral” tissues and (2) an increased liberation of glucose into the circulation from the liver (increased hepatic glucogenesis). There is therefore an extracellular glucose excess and an intracellular glucose deficiency. There is also a decrease in the entry of amino acids into muscle and an increase in lipolysis. These defects result in elevated levels of glucose in the blood and prolonged high blood sugar. Obesity and insulin resistance, the latter of which is generally accompanied by hyperinsulinemia or hyperglycemia, or both, are hallmarks of Type II diabetes [see, e.g., Altshuler et al., U.S. Pat. No. 6,562,574].

Numerous gastrointestinal protein hormones, neurotransmitters and soluble mediators are known to be involved in metabolism. For example, glucagon-like peptide 1 (GLP-1) is an intestinal peptide hormone that plays a critical role in the regulation of the physiological response to feeding. In response to ingestion of a meal, GLP-1 is processed from proglucagon and released into the blood from endocrine L-cells located mainly in the distal small intestine and colon [DiMarchi et al. U.S. Pat. No. 6,583,111 B1]. GLP-1 acts through a G-protein-coupled cell-surface receptor (GLP-1R). GLP-1 stimulates nutrient-induced insulin synthesis and secretion from pancreatic islet cells, thereby lowering blood glucose levels. As such, it is an incretin, a gastrointestinal endocrine factor that is released in response to nutrient intake and stimulates endocrine secretion of the pancreas [DiMarchi et al. U.S. Pat. No. 6,583,111 B1]. GLP-1 also potently inhibits several aspects of digestive function, including gastric emptying, gastric secretion and glucagon secretion. GLP-1 has been shown to dose-dependently inhibit food intake [see, e.g., Turton et al., Nature, 379, 69-72 (1996); Drucker, Gastroenterology, 122, 531-44 (2002)].

Glucose-dependent insulinotropic polypeptide (GIP) is another intestinal peptide hormone that regulates the physiological response to feeding. GIP is released from the small intestine and colon and can interact with a G-protein-coupled receptor, GIP-R. Like GLP-1, GIP is an incretin, stimulating insulin synthesis and secretion after a meal. GIP is essential for the maintenance of glucose homeostasis. GIP also decreases gastric motility and secretion and regulates appetite. In adipose tissue, GIP stimulates fatty acid synthesis, enhances insulin-stimulated incorporation of fatty acids into triglycerides, increases insulin receptor affinity and increases sensitivity of insulin-stimulated glucose transport [Ehses et al., Endocrinology, 144, 4433-45 (2003); Yip and Wolf, Life Sci., 66, 91-103 (2000); Krarup et al., Scand J. Clin. Lab. Invest., 51, 571-79 (1991)].

A third gastrointestinal peptide hormone that regulates metabolism is ghrelin. Ghrelin refers to a family of related peptides of 27 or 28 amino acids that have been isolated in the stomach by a distinct cell type in rats and humans [Kojima et al., Nature, 402, 656-60 (1999); Hosoda et al., J. Biol. Chem., 275, 21995-2000 (2000)]. It is further characterized by having an essential octanoyl ester attached to a serine residue. Ghrelins are known to be potent releasers of growth hormone (GH) in animals and man. They participate in the regulation of energy homeostasis, increase food intake and decrease energy output [Rosicka et al., Physiol. Res., 51, 435-41 (2002)].

The physiological effects of GLP-1, GIP, ghrelin and other gastrointestinal hormones, neurotransmitters or soluble mediators suggest their beneficial use for controlling satiety and treating diabetes and other metabolic disorders. However, a human model experimental system to study how compounds affect both taste transduction and metabolism, particularly diabetes and satiety, is needed to develop new products. Such a model system would be useful for screening for taste modifiers that are also involved in or affect metabolism.

### SUMMARY OF THE INVENTION

This invention generally relates to materials and methods relevant to taste transduction. More particularly, this invention relates to human gastrointestinal cells that comprise or are capable of expressing endogenous taste signaling proteins. Even more particularly, this invention relates to human gastrointestinal cells that comprise or are capable of expressing endogenous taste signaling proteins as well as hormones, neurotransmitters or soluble mediators of the gastrointestinal tract that are involved in or affect metabolism, digestion and appetite. This invention further relates to the use of these human cells or their membranes to study how compounds affect taste transduction and/or metabolism, digestion and appetite, including effects on satiety, emesis and diabetes.

In some embodiments, the invention provides a method of testing whether a compound affects taste transduction comprising: (a) contacting a human gastrointestinal cell or its membrane with the compound, wherein the cell or membrane comprises one or more taste signaling proteins; and (b) evaluating the effect of the compound on the cell or membrane.

In some embodiments, the invention provides a method of identifying a modulator of taste transduction comprising: (a) contacting a human gastrointestinal cell or its membrane with a tastant, wherein the cell or membrane comprises one or more taste signaling proteins; (b) contacting the cell or its membrane with a compound; and (c) evaluating the compound's effect on tastant-mediated taste transduction, wherein a compound that alters tastant-mediated taste transduction is a modulator.

In some embodiments, the invention provides a method of identifying a mimic of a tastant comprising: (a) contacting a human gastrointestinal cell or its membrane with a tastant, wherein the cell or membrane comprises one or more taste signaling proteins; (b) evaluating the effect of the tastant on the cell or membrane; (c) in a separate experiment, contacting the cell or its membrane with a compound; (d) evaluating the effect of the compound on the cell or membrane; and (e) comparing the effect of the tastant with the effect of the compound; wherein a compound that affects the cell or membrane in the same manner as the tastant is a mimic of the tastant.

In some embodiments, the invention provides a method of testing whether a compound affects both taste transduction and signal transduction of one or more gastrointestinal protein hormones, neurotransmitters or soluble mediators involved in metabolism comprising: (a) contacting a human gastrointestinal cell with the compound, wherein the cell comprises one or more taste signaling proteins and is also capable of synthesizing or secreting the one or more gastrointestinal protein hormones, neurotransmitters or soluble mediators; (b) evaluating the effect of the compound on the one or more taste signaling proteins; and (c) evaluating the effect of the compound on the cell's synthesis or secretion of the one or more gastrointestinal protein hormones, neurotransmitters or soluble mediators.

In some embodiments, the invention provides a method of testing whether a modulator of taste transduction also affects signal transduction of one or more gastrointestinal protein hormones, neurotransmitters or soluble mediators involved in metabolism comprising: (a) contacting a human gastrointestinal cell that comprises one or more taste signaling proteins and is also capable of synthesizing or secreting the one or more gastrointestinal protein hormones, neurotransmitters or soluble mediators with the modulator; and (b) evaluating the effect of the modulator on the cell's synthesis or secretion of the one or more gastrointestinal protein hormones, neurotransmitters or soluble mediators.

In some embodiments, the invention provides a method of testing whether a mimic of a tastant also affects signal transduction of one or more gastrointestinal protein hormones, neurotransmitters or soluble mediators involved in metabolism comprising: (a) contacting a human gastrointestinal cell that comprises one or more taste signaling proteins and is also capable of synthesizing or secreting the one or more gastrointestinal protein hormones, neurotransmitters or soluble mediators with the mimic; and (b) evaluating the effect of the mimic on the cell's synthesis or secretion of the one or more gastrointestinal protein hormones, neurotransmitters or soluble mediators.

In some embodiments, the human gastrointestinal cells are derived from endocrine cells. In some embodiments, the cells are derived from endocrine L-cells. In some embodiments, the cells are NCI-H716 cells (ATCC No. CCL-251).

In some embodiments, the one or more gastrointestinal protein hormones, neurotransmitters or soluble mediators involved in metabolism are selected from a group comprising: GLP-1, GLP-2, GIP, ghrelin, serotonin, epineprine, norepineprine and nitrogen oxide.

In some embodiments, the human gastrointestinal cells comprise at least one of the following taste signaling proteins: T1R1, T1R2, T1R3, T1R4, T2R, Trpm5, PDE1A, PLCβ2, Gy13, Gβ3, inositol trisphosphate receptor type 3, adenylyl cyclase isoform 8, gustducin α and transducin α.

In some embodiments, the tastant is a molecule from food or beverage, a medicament, a component of the medicament, a breakdown product of the component of the medicament, a preservative, a nutritional supplement, a medical or dental composition, an oral film, a cosmetic, a metallic salt, a composition used in pest control, soap, shampoo, toothpaste, mouthwash, niouthrinse, denture adhesive, glue on the surface of stamps or glue on the surface of envelopes. In some embodiments, the component of the medicament is a vehicle for the medicament.

In some embodiments, the effect of the compound, modulator or mimic comprises an increase in the human gastrointestinal cell's synthesis or secretion of the one or more gastrointestinal protein hormones, neurotransmitters or soluble mediators. In some embodiments, the effect of the compound, modulator or mimic comprises a decrease in the human gastrointestinal cell's synthesis or secretion of the one or more gastrointestinal protein hormones, neurotransmitters or soluble mediators.

In some embodiments, the effect of the compound, tastant, modulator or mimic is on a signaling molecule. In some embodiments, the signaling molecule is selected from a group comprising: cAMP, cGMP, IP₃, DAG, PDE and Ca^{2+.}

In some embodiments, the effect of the compound, tastant, modulator or mimic is evaluated by measuring levels of ions, phosphorylation, dephosphorylation or transcription. In some embodiments, the effect of the compound, tastant, modulator or mimic is evaluated by detecting changes in levels of ions, phosphorylation, dephosphorylation or transcription.

In some embodiments, the effect of the compound, tastant, modulator or mimic is evaluated by measuring levels of CAMP, cGMP, IP₃, DAG, PDE or Ca²⁺. In some embodiments, the effect is evaluated by detecting changes in levels of CAMP, cGMP, IP₃, DAG, PDE or Ca²⁺.

In some embodiments, the effect of the compound, tastant, modulator or mimic is evaluated using an immunoassay or bioassay. In some embodiments, the immunoassay or bioassay detects the one or more taste signaling proteins. In some embodiments, the immunoassay or bioassay detects the synthesis or secretion of the one or more gastrointestinal protein hormones, neurotransmitters or soluble mediators.

### BRIEF DESCRIPTION OF THE DRAWINGS

FIG. 1 depicts immunofluorescence analysis of GLP-1 and gustducin a using NCI-H716 cells.

FIGS. 2A and 2B depict RT-PCR analysis of human enteroendocrine cells expressing TAS1Rs and TAS2Rs, respectively.

FIG. 3 compares RT-PCR analysis of NCI-H716 cells and rat lingual-epithelial cells. The RT-PCR reaction was performed using published primer sequences for gustducin α, T1R1, T1R2, T1R3, Trpm5, PDE1A and PLCβ2.

FIGS. 4A, 4B and 4C are graphs depicting the results of [³⁵S]GTPγS binding assays using NCI-H716 cells. The cells were stimulated with dextromethorphan in the presence and absence of transducin, and the binding of GTPγS to G proteins was measured. Fractions S1, P1 and P2 refer to the supernatant and pellets prepared from the cell membranes via centrifugation. S1 is the first supernatant, P1 is the first pellet and P2 is the high-speed pellet from the S1 fraction. NSB refers to non-specific binding.

FIGS. 5A, 5B and 5C are graphs depicting the results of [³⁵S]GTPγS binding assays using NCI-H716 cells. The cells were stimulated with doxylamine in the presence and absence of transducin, and the binding of GTPγS to G proteins was measured. Fractions S1, P1 and P2 refer to the supernatant and pellets prepared from the cell membranes via centrifugation. S1 is the first supernatant, P1 is the first pellet and P2 is the high-speed pellet from the S1 fraction. NSB refers to non-specific binding.

FIGS. 6A and 6B are saturation curves. FIG. 6A depicts dependence and saturation where increasing amounts of NCI-H716 cell membranes were exposed to 10 mM dextromethorphan. FIG. 6B depicts dependence and saturation where 2 μg of NCI-H716 cell membranes was exposed to increasing amounts of dextromethorphan.

### DETAILED DESCRIPTION OF THE INVENTION

Definitions and General Techniques

Unless defined otherwise, all technical and scientific terms used herein have the same meaning as commonly understood by one of ordinary skill in the art. In case of conflict, the present application including the definitions will control. Also, unless otherwise required by context, singular terms shall include pluralities, and plural terms shall include the singular. All publications, patents and other references mentioned herein are incorporated by reference.

Although methods and materials similar or equivalent to those described herein can be used in practice or testing of the present invention, suitable methods and materials are described below. The materials, methods and examples are illustrative only, and are not intended to be limiting. Other features and advantages of the invention will be apparent from the detailed description and from the claims.

Throughout this specification and claims, the word “comprise,” or variations such as “comprises” or “comprising,” will be understood to imply the inclusion of a stated integer or group of integers but not the exclusion of any other integer or group of integers.

In order to further define this invention, the following terms and definitions are herein provided.

The terms “inhibitor,” “activator,” and “modulator” are used interchangeably to refer to inhibitory, activating or modulating molecules identified using assays for signal transduction, e.g., ligands, agonists, antagonists and their homologs and mimetics. Inhibitors include compounds that block, decrease, prevent, delay activation of, inactivate, desensitize or down-regulate signal transduction, e.g., antagonists. Inhibitors include compounds that, e.g., bind to components of signal transduction to partially or totally block stimulation of signal transduction. Activators include compounds that stimulate, increase, initiate, activate, facilitate, enhance activation of, sensitize or up-regulate signal transduction, e.g., agonists. Activators include compounds that, e.g., bind to components of signal transduction to stimulate signal transduction. Modulators include inhibitors and activators. Modulators also include compounds that, e.g., alter the interaction of a receptor with: extracellular proteins that bind activators or inhibitors; G-proteins; kinases (e.g., homologs of rhodopsin kinase and beta adrenergic receptor kinases that are involved in deactivation and desensitization of a receptor); and arrestin-like proteins, which also deactivate and desensitize receptors. Modulators include naturally occurring and synthetic ligands, antagonists, agonists, small chemical molecules and the like.

The terms “polypeptide,”. “peptide” and “protein” are used interchangeably herein to refer to a polymer of amino acid residues. The terms apply to amino acid polymers in which one or more amino acid residues are artificial chemical mimetics of corresponding naturally occurring amino acids. The terms apply to naturally occurring amino acid polymers and non-naturally occurring amino acid polymers.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled with, e.g., isotopes, chromophores, lumiphores or chromogens, or indirectly labeled with, e.g., biotin, to which a streptavidin complex may later bind. By assaying for the presence or absence of the probe, one can detect the presence or absence of the select sequence or subsequence.

Oligonucleotides that are not commercially available can be chemically synthesized using methods known to those of skill in the art. These methods include the solid phase phosphoramidite triester method first described by Beaucage & Caruthers, Tetrahedron Letts., 22, 1859-62 (1981), using an automated synthesizer, as described in Van Devanter et. al., Nucleic Acids Res., 12, 6159-68 (1984). Purification of oligonucleotides is by either native acrylamide gel electrophoresis or by anion-exchange HPLC as described in Pearson & Reanier, J. Chrom., 255, 137-49 (1983).

The term “recombinant” when used with reference to, e.g., a cell, nucleic acid, protein or vector, indicates that the cell, nucleic acid, protein or vector has been modified by the introduction of a heterologous nucleic acid or protein or the alteration of a native nucleic acid or protein, or that the cell is derived from a cell so modified. Thus, for example, recombinant cells-express genes that are not found within the native (non-recombinant) form of the cell or express native genes that are otherwise abnormally expressed, underexpressed or not expressed at all.

The word “tastant” is generally understood to include substances having a taste quality. It will be obvious to those skilled in the art that the word “tastant” is also applicable to (1) substances largely without taste that bind to taste bud membranes; and (2) other substances that are involved in taste bud function whose action depends upon binding to taste receptors.

The word “T2R” as used in this application refers to all receptors in the taste receptor 2 family.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Gene expression of a taste signaling protein or a gastrointestinal protein that regulates metabolism can be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like [see, e.g., Gunthand et al., AIDS Res. Hum. Retroviruses, 14, 869-76 (1998); Kozal et al., Nat. Med., 2, 753-59 (1996); Matson et al., Anal. Biochem., 224, 110-16 (1995); Lockhart et al., Nat. Biotechnol., 14, 1675-80 (1996); Gingeras et al., Genome Res., 8, 435-48 (1998); Hacia et. al., Nucleic Acids Res., 26, 3865-66 (1998)].

Cells

In one aspect the present invention relates to human gastrointestinal cells that comprise or are capable of expressing endogenous taste signaling proteins. In some embodiments of the invention, the cells are endocrine cells. In some embodiments, the cells are endocrine L cells. In some embodiments of the invention, the cells are NCI-H716 cells. (ATCC Number CCL-251).

In some embodiments the cells comprise at least one of the following taste signaling proteins: T1R1, T1R2, T1R3, T1R4, T2R, Trpm5, PDE1A, PLCβ2, Gγ13, Gβ3, inositol trisphosphate receptor type 3, adenylyl cyclase isoform 8, gustducin α and transducin α. In some embodiments the human cells comprise at least three of the above taste signaling proteins.

In some embodiments, in addition to taste signaling proteins, the human gastrointestinal cells also comprise or are capable of expressing at least one gastrointestinal protein hormone, neurotransmitter or soluble mediator involved in metabolism. In some embodiments the gastrointestinal protein hormone, neurotransmitter or soluble mediator involved in metabolism is selected from the group comprising GLP-1, GLP-2, GIP, ghrelin, serotonin, epinephrine, norepinephrine and nitrogen oxide. Human cells that comprise or are capable of expressing both taste signaling proteins and gastrointestinal protein hormones, neurotransmitters or soluble mediators involved in metabolism would be useful in the area of taste as well as metabolic research, e.g., research regarding metabolic disorders such as diabetes, emesis and satiety-related disorders.

Whether the human cells comprise or are capable of expressing taste signaling proteins and/or gastrointestinal protein hormones, neurotransmitters or soluble mediators involved in metabolism can be determined by assays known to a person of skill in the art, e.g., immunoassays, bioassays, RNA in situ, Northern blot, GTPγS dinding assay, trypsin sensitivity, RNAse Protection or RT-PCR (see Example 2). Further, proteins associated with taste signaling and gastrointestinal protein hormones, neurotransmitters or soluble mediators involved in metabolism are known to a person of skill in the art. Their amino acid sequences and the sequences of DNAs encoding them can be easily obtained, e.g., via the GenBank database.

In some embodiments the human cells comprise or are capable of expressing at least one recombinant taste signaling protein in addition to an endogenous taste signaling protein. In some embodiments the human cells comprise or are capable of expressing at least one recombinant gastrointestinal protein involved in metabolism in addition to an endogenous taste signaling protein.

Assays

This invention further provides methods of using human gastrointestinal cells or cell membranes to test for compounds that interact with taste signaling proteins and/or gastrointestinal protein hormones, neurotransmitters, or soluble mediators involved in metabolism, digestion or appetite either directly or indirectly, e.g., tastants, activators, inhibitors, enhancers, stimulators, agonists, antagonists, modulators and mimics. This invention provides assays for taste modulation wherein the taste signaling protein(s) and/or gastrointestinal protein hormone(s), neurotransmitter(s), or soluble mediator(s) involved in metabolism, digestion or appetite acts as a direct or indirect reporter molecule(s) for the effect of a compound on signal transduction. The human gastrointestinal cells of this invention or their membranes can be used for such assays, e.g., to measure or detect changes in levels of the one or more taste signaling proteins and/or the one or more gastrointestinal protein hormones, neurotransmitters or soluble mediators synthesized or secreted by the cell, or to detect or measure changes in membrane potential, current flow, ion flux, transcription, phosphorylation, dephosphorylation, signal transduction, receptor-ligand interactions, second messenger concentrations, etc.

In some embodiments the invention provides a method of testing whether a compound affects taste transduction comprising: (a) contacting a human gastrointestinal cell or its membrane with the compound, wherein the cell or membrane comprises one or more taste signaling proteins; and (b) evaluating the effect of the compound on the cell or membrane.

In some embodiments the invention provides a method of testing whether a compound affects both taste transduction and signal transduction of one or more gastrointestinal protein hormones, neurotransmitters or soluble mediators involved in metabolism comprising: (a) contacting a human gastrointestinal cell with the compound, wherein the cell comprises one or more taste signaling proteins and is also capable of synthesizing or secreting the one or more gastrointestinal protein hormones, neurotransmitters or soluble mediators; (b) evaluating the effect of the compound on the one or more taste signaling proteins; and (c) evaluating the effect of the compound on the cell's synthesis or secretion of the one or more gastrointestinal protein hormones, neurotransmitters or soluble mediators.

In some embodiments the invention provides a method of identifying a modulator of taste transduction comprising: (a) contacting a human gastrointestinal cell or its membrane with a tastant, wherein the cell or membrane comprises one or more taste signaling proteins; (b) contacting the cell or its membrane with a compound; and (c) evaluating the compound's effect on tastant-mediated taste transduction, wherein a compound that alters tastant-mediated taste transduction is a modulator.

In some embodiments the invention provides a method of testing whether a modulator of taste transduction also affects signal transduction of one or more gastrointestinal protein hormones, neurotransmitters or soluble mediators involved in metabolism comprising: (a) contacting a human gastrointestinal cell that comprises one or more taste signaling proteins and is also capable of synthesizing or secreting the one or more gastrointestinal protein hormones, neurotransmitters or soluble mediators with the modulator; and (b) evaluating the effect of the modulator on the cell's synthesis or secretion of the one or more gastrointestinal protein hormones, neurotransmitters or soluble mediators.

In some embodiments the invention provides a method of identifying a mimic of a tastant comprising: (a) contacting a human gastrointestinal cell or its membrane with a tastant, wherein the cell or membrane comprises one or more taste signaling proteins; (b) evaluating the effect of the tastant on the cell or membrane; (c) in a separate experiment, contacting the cell or its membrane with a compound; (d) evaluating the effect of the compound on the cell or membrane; and (e) comparing the effect of the tastant with the effect of the compound; wherein a compound that affects the cell or membrane in the same manner as the tastant is a mimic of the tastant.

In some embodiments the invention provides a method of testing whether a mimic of a tastant also affects signal transduction of one or more gastrointestinal protein hormones, neurotransmitters or soluble mediators involved in metabolism comprising: (a) contacting a human gastrointestinal cell that comprises one or more taste signaling proteins and is also capable of synthesizing or secreting the one or more gastrointestinal protein hormones, neurotransmitters or soluble mediators with the mimic; and (b) evaluating the effect of the mimic on the cell's synthesis or secretion of the one or more gastrointestinal protein hormones, neurotransmitters or soluble mediators.

In some embodiments the human gastrointestinal cells or their membranes can be used in a direct reporter assay to detect whether a compound, tastant, modulator or mimic affects taste transduction and/or signal transduction of one or more gastrointestinal protein hormones, neurotransmitters or soluble mediators involved in metabolism.

In some embodiments the human gastrointestinal cells or their membranes can be used in an indirect reporter assay to detect whether a compound, tastant, modulator or mimic affects taste transduction and/or signal transduction of one or more gastrointestinal protein hormones, neurotransmitters or soluble mediators involved in metabolism [see, e.g., Mistili & Spector, Nature Biotechnology, 15, 961-64 (1997)].

In some embodiments the human gastrointestinal cells or their membranes can be used to assay the binding of a compound, tastant, modulator or mimic that affects signal transduction by studying, e.g., changes in spectroscopic characteristics (e.g., fluorescence, absorbance, refractive index) or hydrodynamic (e.g., shape), chromatographic or solubility properties.

In some embodiments the human gastrointestinal cells or their membranes can be used to examine the effect of a compound, tastant, modulator or mimic on interactions between a receptor and a G protein. For example, binding of a G protein to a receptor or release of the G protein from the receptor can be examined. In the absence of GTP, an activator will lead to the formation of a tight complex of all three subunits of the G protein with the receptor. This complex can be detected in a variety of ways, as noted above. Such an assay can be modified to search for inhibitors of taste transduction or inhibitors of signal transduction of one or more gastrointestinal protein hormones, neurotransmitters or soluble mediators. For example, an activator could be added to the receptor and G protein in the absence of GTP such that a tight complex forms, which could then be screened for inhibitors by studying dissociation of the receptor-G protein complex. In the presence of GTP, release of the alpha subunit of the G protein from the other two G protein subunits serves as a criterion of activation.

An activated or inhibited G protein will in turn influence downstream steps of the signal transduction pathway, affecting, e.g., the properties of target enzymes, channels and other effectors. Examples of downstream steps include activation of cGMP phosphodiesterase by transducin in the visual system, adenylyl cyclase by the stimulatory G protein, phospholipase C by G_{q} and other cognate G proteins, and modulation of diverse channels by Gᵢ and other G proteins. In some embodiments, the human gastrointestinal cells or their membranes can be used to examine the effect of a compound, tastant, modulator or mimic on intermediate steps of signal transduction, such as the generation of diacyl glycerol and IP₃ by phospholipase C and, in turn, calcium mobilization by IP₃. In some embodiments, the compound, tastant, modulator or mimic may act directly on, e.g., the G protein, affecting downstream events indirectly. In some embodiments, the compound, tastant, modulator or mimic may directly affect the downstream effector. For a general review and methods of assaying taste signal transduction and gastrointestinal protein hormone signal transduction, see, e.g., Methods in Enzymology, vols. 237 and 238 (1994) and volume 96 (1983); Bourne et al., Nature, 10, 117-27 (1991); Bourne et al., Nature, 348, 125-32 (1990); Pitcher et al., Annu. Rev. Biochem., 67, 653-92 (1998); Brubaker et al., Receptors Channels, 8, 179-88 (2002); Kojima et al., Curr. Opin. Pharmacol., 2, 665-68 (2002); Bold et al., Arch Surg., 128, 1268-73 (1993).

In some embodiments the effect of the compound, tastant, modulator or mimic comprises an increase or a decrease in the human gastrointestinal cell's synthesis or secretion of the one or more taste signaling proteins. In some embodiments the effect of the compound, tastant, modulator or mimic also comprises an increase or a decrease in the human gastrointestinal cell's synthesis or secretion of the one or more gastrointestinal protein hormones, neurotransmitters or soluble mediators.

The effects of the compounds, tastants, modulators or mimics on taste signaling polypeptides and/or gastrointestinal protein hormones, neurotransmitters or soluble mediators can be examined by performing any of the assays described above. Any suitable physiological change that affects these signaling pathways can be used to assess the influence of a compound on the cells of this invention.

The effects of compounds, tastants, modulators or mimics on signal transduction in any of the above assays may be detected or measured in a variety of ways. For example, one can detect or measure effects such as transmitter release, hormone release, transcriptional changes to both known and uncharacterized genetic markers (e.g., northern blots), changes in cell metabolism such as cell growth or pH changes, ion flux, phosphorylation, dephosphorylation, and changes in intracellular second messengers such as Ca²⁺, IP₃, DAG, PDE, cGMP or cAMP. Changes in second messenger levels can be optionally measured using, e.g., fluorescent Ca²⁺ indicator dyes and fluorometric imaging.

In some embodiments the effects of the compound, tastant, modulator or mimic on G-protein-coupled receptors can be measured by using cells that are loaded with ion- or voltage-sensitive dyes, which report receptor activity. Assays that examine the activity of such proteins can also use known agonists and antagonists for other G-protein-coupled receptors as negative or positive controls to assess the activity of the tested compounds. To identify modulatory compounds, changes in the level of ions in the cytoplasm or membrane voltage can be monitored using an ion-sensitive or membrane-voltage fluorescent indicator, respectively. Among the ion-sensitive indicators and voltage probes that may be employed are those sold by Molecular Probes or Invitrogen. For G-protein-coupled receptors, lax G-proteins such as Ga15 and Ga16 can be used in the assay of choice [Wilkie et al., Proc. Natl. Acad. Sci., USA, 88, 10049-53 (1991)]. Such lax G-proteins allow coupling of a wide range of receptors.

In some embodiments the effects of the compound, tastant, modulator or mimic can be measured by calculating changes in cytoplasmic calcium ion levels. In some embodiments, levels of second messengers such as IP₃ can be measured to assess G-protein-coupled receptor function [Berridge & Irvine, Nature, 312, 315-21 (1984)]. Cells expressing such G-protein-coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

In some embodiments the effects of the compound, tastant, modulator or mimic can be measured by determining the activity of proteins which, when activated, result in a change in the level of intracellular cyclic nucleotides, e.g., cAMP or cGMP, by activating or inhibiting enzymes such as adenylyl cyclase. There are cyclic nucleotide-gated ion channels, e.g., rod photoreceptor cell channels and olfactory neuron channels that are permeable to cations upon activation by binding of cAMP or cGMP [see, e.g., Altenhofen et al., Proc. Natl. Acad. Sci. U.S.A., 88, 9868-72 (1991); Dhallan et al., Nature, 347, 184-87 (1990)]. In cases where activation of the protein results in a decrease in cyclic nucleotide levels, it may be preferable to expose the cells to agents that increase intracellular cyclic nucleotide levels, e.g., forskolin, prior to adding a compound to the cells in the assay.

In some embodiments the effects of the compound, tastant, modulator or mimic can be measured by calculating changes in intracellular cAMP or cGMP levels using immunoassays or bioassays [Simon, J. Biol. Chem., 270, 15175-80 (1995); Felley-Bosco et al., Am. J. Resp. Cell and Mol. Biol., 11, 159-64 (1994); U.S. Pat. No. 4,115,538].

In some embodiments the effects of the compound, tastant, modulator or mimic can be measured by examining phosphatidyl inositol (PI) hydrolysis according to U.S. Pat. No. 5,436,128.

In some embodiments the effects of the compound, tastant, modulator or mimic on signal transduction can be measured by calculating transcription levels. The human cell or its membrane containing the protein of interest may be contacted with a compound, tastant, modulator or mimic for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots, or polypeptide products may be identified using immunoassays or bioassays. Alternatively, transcription-based assays using reporter gene(s) may be used as described in U.S. Pat. No. 5,436,128. The reporter gene(s) can be, e.g., chloramphenicol acetyltransferase, firefly luciferase, bacterial luciferase, β-galactosidase and alkaline phosphatase. Furthermore, the protein of interest can act as an indirect reporter via attachment to a second reporter such as green fluorescent protein [see, e.g., Mistili & Spector, Nature Biotechnology, 15, 961-64 (1997)].

In some embodiments, the amount of transcription is then compared to the amount of transcription in the same cell in the absence of the compound, tastant, modulator or mimic. Alternatively, the amount of transcription may be compared with the amount of transcription in a substantially identical cell that lacks the protein of interest. For example, a substantially identical cell may be derived from the same cells from which the recombinant cell was prepared but which had not been modified by introduction of heterologous DNA. Any difference in the amount of transcription indicates that the compound, tastant, modulator or mimic has in some manner altered the activity of the protein of interest. In some embodiments, the compound, tastant, modulator or mimic is administered in combination with a known agonist or antagonist of transcription, to determine whether the compound, tastant, modulator or mimic can alter the activity of the agonist or antagonist.

The compounds, tastants, modulators or mimics tested can be any small chemical compound, or a biological entity, such as a protein, amino acid, sugar, nucleic acid or lipid. Alternatively, the compounds can be variants of taste signaling proteins. Typically, compounds will be small chemical molecules and peptides. Essentially any chemical compound can be used as a potential tastant, modulator or mimic in the assays of the invention although most often compounds dissolved in aqueous or organic (especially DMSO-based) solutions are used. The assays can be used to screen large chemical libraries by automating the assay steps (e.g., in microtiter formats on microtiter plates in robotic assays).

In some embodiments the compound tested is a tastant. Examples of tastants include a molecule from a food, a beverage, a medicament, a component of the medicament, a breakdown product of the component of the medicament, a preservative or a nutritional supplement. In some embodiments, the component of the medicament is a vehicle for the medicament. Other examples of tastants include a molecule from a medical or dental composition, such as a contrast material or a local oral anesthetic, or from a cosmetic, such as a face cream or lipstick. The tastant can also be a metallic salt, an oral film, or a molecule from any composition that may contact taste membranes. Examples include, but are not limited to, soap, shampoo, toothpaste, mouthwash, mouthrinse, denture adhesive, glue on the surface of stamps, glue on the surface of envelopes, or a composition used in pest control, such as rat or cockroach poison.

In some embodiments, high-throughput screening methods involve providing a combinatorial chemical or peptide library containing a large number of compounds that potentially affect taste transduction. Such “combinatorial libraries” or “ligand libraries” are then screened in one or more assays, as described herein, to identify those library members (particular chemical species or subclasses) that have the desired characteristic activity.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries [see, e.g., U.S. Pat. No. 5,010,175; Furka, Int. J. Pept. Prot. Res., 37, 487-93 (1991); Houghton et al., Nature, 354, 84-88 (1991)]. Other methods for generating chemically diverse libraries can also be used [see, e.g., PCT Publication No. WO 91/19735; PCT Publication No. WO 93/20242; PCT Publication No. WO 92/00091; U.S. Pat. No. 5,288,514; Hobbs et al., Proc. Nat. Acad. Sci. USA, 90, 6909-13 (1993); Hagihara et al., J. Amer. Chem. Soc., 114, 6568 (1992); Hirschmann et al., J. Amer. Chem. Soc., 114, 9217-18 (1992); Chen et al., J. Amer. Chem. Soc., 116, 2661 (1994); Cho et al., Science, 261, 1303 (1993); Campbell et al., J. Org. Chem., 59, 658 (1994); Ausubel, Berger and Sambrook, all supra; U.S. Pat. No. 5,539,083; Vaughn et al., Nature Biotechnology, 14, 309-14 (1996); PCT US96/10287; Liang et al., Science, 274, 1520-22 (1996); U.S. Pat. No. 5,593,853; Baum, C&EN, January 18, page 33 (1993); U.S. Pat. No. 5,569,588; U.S. Pat. No. 5,549,974; U.S. Pat. No. 5,525,735; U.S. Pat. No. 5,519,134; U.S. Pat. No. 5,506,337 and U.S. Pat. No. 5,288,514].

Devices for the preparation of combinatorial libraries are commercially available [see, e.g., 357 MPS, 390 MPS, Advanced Chem Tech, Louisville Ky., Symphony, Rainin, Wobum, Mass., 433A Applied Biosystems, Foster City, Calif., 9050 Plus, Millipore, Bedford, Mass.]. Also, numerous combinatorial libraries are commercially available [see, e.g., ComGenex, Princeton, N.J., Tripos, Inc., St. Louis, Mo., 3D Pharmaceuticals, Exton, Pa., Martek Biosciences, Columbia, Md., etc.].

In one embodiment, the human gastrointestinal cells or their membranes may be used to prepare a library of compound signal transduction profiles. This method involves contacting the cells or their membranes with a compound, wherein the cells or membranes comprise one or more taste signaling proteins and/or one or more gastrointestinal protein hormones, neurotransmitters or soluble mediators, and evaluating the effect of the compound on the cells or membranes. In some embodiments, this method includes evaluating the effect of the compound on one or more taste signaling molecules and/or one or more gastrointestinal protein hormones, neurotransmitters or soluble mediators. The resulting information constitutes a signal transduction profile of the compound. Profiles of this type may be collected for a number of compounds to create a library of signal transduction profiles. Test agents may then be similarly profiled and compared to such a library to identify agent profiles that match profiles in the library. This technique may be used to identify potential substitutes for or mimics of compounds in the library. In some embodiments, the human gastrointestinal cells or their membranes may be used to prepare a library of tastant signal transduction profiles, and the library may be used to identify tastant substitutes.

Each of the foregoing steps can be performed in a variety of ways. A skilled artisan can readily adapt the human cell or cell membrane of the present invention for use in any taste signaling assay or assay to test gastrointestinal protein hormones, neurotransmitters or soluble mediators involved in metabolism.

In order that this invention may be better understood, the following examples are set forth. These examples are for purposes of illustration only and are not to be construed as limiting the scope of the invention in any manner.

### EXAMPLE 1

### Immunofluorescence and RT-PCR Assays for GLP-1

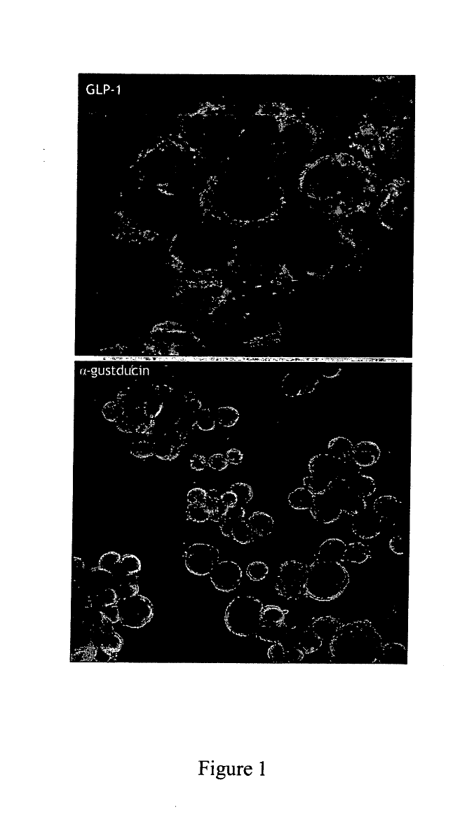

Cells were grown on matrigel-coated cover slips and grown to confluent monolayers in 12-well plates at 37° C. They were fixed in 4% paraformaldehyde in phosphate-buffered saline (PBS) and incubated with the primary antiserum (rabbit anti-alpha gustducin, 1:150; Santa Cruz Biotechnology, and rabbit anti-GLP-1, Phoenix) overnight at 4° C. following permeabilization with 0.4% Triton-X in PBS for 10 minutes and blocking for 1 hour at room temperature. Following three washing steps with blocking buffer, the appropriate secondary antibody was applied (AlexaFluor 488 anti-rabbit immunoglobulin, 1:1000; Molecular Probes) for 1 hour at room temperature. After three washing steps, the cells were fixed in Vectashield medium. As shown in FIG. 1, NCI-H716 cells contain GLP-1 and gustducin α.

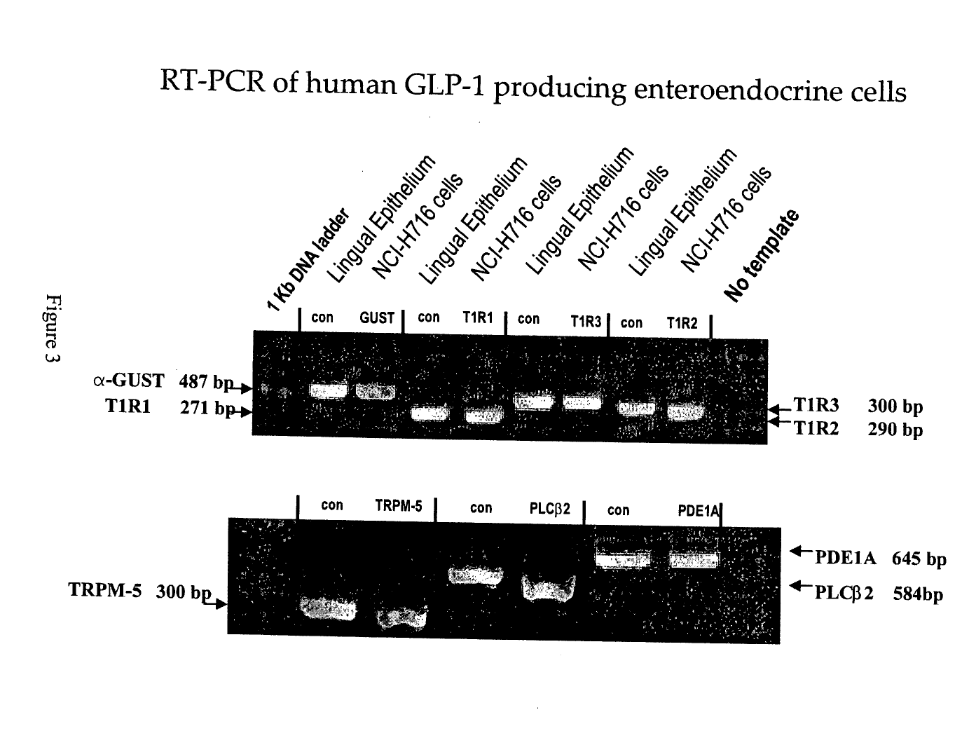

RT-PCR RNA isolation from cells was done using standard methodology. The RT-PCR reaction was performed in a volume of 50 μl in a Peltier thermal cycler (PTC-225 DNA Engine Tetrad Cycler; MJ Research), using published primer sequences (Integrated DNA Technologies). Reverse transcription was performed at 50° C. for 30 minutes; after an initial activation step at 95° C. for 15 minutes, the PCR conditions were as follows: denaturing at 94° C. for 1 minute, annealing at 55° C. for 1 minute and extension at 72° C. for 1 minute for 40 cycles, followed by a final extension step at 72° C. for 10 minutes. In each procedure, two negative controls were included, where water was substituted for the omitted reverse transcriptase or template. The control was RNA isolated from rat lingual epithelium. PCR products were resolved in 2% agarose gel with ethidium bromide and visualized under UV light. As shown in FIG. 3, NCI-H716 cells contain the following taste signaling proteins: gustducin α, T1R1, T1R2, T1R3, Trpm-5, PDE1A and PLCβ2.

### EXAMPLE 2

### RT-PCR Assays for TAS1R and TAS2R Receptors

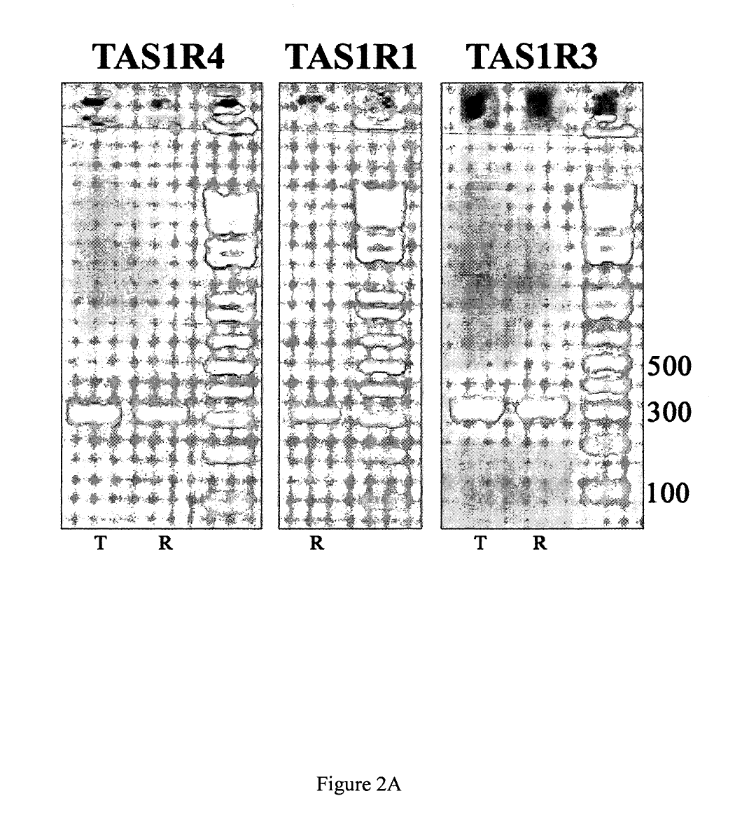

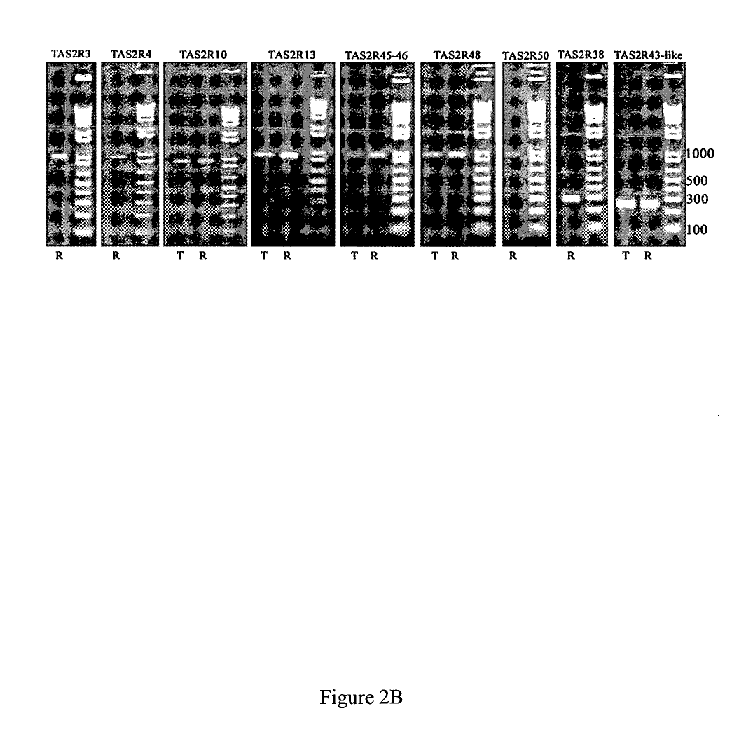

Total RNA isolated from NCI-H716 cells was reverse-transcribed using either oligo-dT (lanes marked “T” in FIGS. 2A and 2B) or random primers (lanes marked “R” in FIGS. 2A and 2B) to generate cDNA for PCR. PCR was carried out with different specific primer sets to amplify fragments or full-length transcripts of individual members of Taste Receptor family 1 (TAS1Rs, see FIG. 2A) and of Taste Receptor family 2 (TAS2Rs, see FIG. 2B). Because TAS2R43 and TAS2R44 are quite similar and TAS2R45 and TAS2R46 are also quite similar, in these cases degenerate primer pairs were used to amplify full length TAS2R45/TAS2R46 receptors (TAS2R45-46, see FIG. 2B) and an ˜300 bp fragment of TAS2R43/TAS2R44 receptors (TAS2R43-like, see FIG. 2B). PCR products were electrophoresed through 2% agarose gels followed by ethidium bromide staining to visualize the amplified DNAs. To determine the size of the PCR products molecular weight markers (1 Kb+ ladder; Invitrogen) were included (right lane of each gel). As a positive control for reverse transcription, β-actin was amplified by PCR from oligo-dT- or random-primed cDNA (not shown). To ensure that PCR products with receptor-specific primer sets did not represent amplification of contaminating DNA (cDNA or genomic DNA), negative controls were included in which PCR was performed with no added DNA template or with template from a cDNA reverse transcription reaction in which reverse transcriptase had been omitted (data not shown).

RT-PCR was used to detect the presence of TAS1R and TAS2R taste receptor transcripts in NCI-H716 cells. NCI-H716 cells grown in a 100 mm culture dish to ˜60% confluence were harvested by trituration, followed by low-speed centrifugation and removal of the DMEM culture media. After rinsing the cells with PBS, the cells were resuspended in RNA-later (Ambion). Total RNA was then isolated using the Absolutely RNA Microprep Kit (Stratagene). This preparation includes a DNAse1 step to eliminate traces of contaminating genomic DNA. The final RNA pellet was dissolved in Diethyl Pyrocarbonate (DEPC)-treated water. To make cDNA, 100 units of Superscript-II (Invitrogen) was used to reverse transcribe 1 μg of total RNA in a 50 μl reaction containing either oligo-dT or random hexamers, at 42° C. for 60 minutes (following Invitrogen's Superscript First Strand Synthesis System protocol). As a negative control a reaction was set up with RNA and with oligo-dT primer in which Superscript-II reverse transcriptase (RT) was omitted.

For PCR, 1/100th of each RT reaction was used as template in 25 μl of mix that included 1×PCR buffer (20 mM Tris pH 8.4, 50 mM KCl, 1.5 mM MgCl2, 200 μM of each DNTP), 40 pmol of each of the forward and reverse gene-specific primers, and 1 unit of Platinum Taq DNA polymerase (Invitrogen). PCR was carried out at 94° C. for 3 minutes, followed by 40 cycles at 94° C. for 30 seconds, 56° C. for 30 seconds, and 72° C. for 1 minute. The last step was followed by a 2 minute extension step t 72° C. The PCR products were resolved on 2% agarose els and visualized by ethidium bromide staining.

PCR products of the expected size were obtained for TAS1R1 and TAS1R3 (which when heterologously expressed form a umami/amino acid receptor) and for a T1R-like orphan receptor that we have named TAS1R4 (FIG. 2A). PCR products were obtained using gene-specific primers for 7 different presumptive bitter receptors of the TAS2R family (TAS2R3, TAS2R4, TAS2R10, TAS2R13, TAS2R38, and TAS2R48), and PCR products were obtained using degenerate primers designed to amplify members of the subfamily (TAS2R43, TAS2R44, TAS2R45, and TAS2R46) (FIG. 2B).

### EXAMPLE 3

### Radiometric [³⁵S] GTPγS Binding Assay

[³⁵S] GTPγS binding assays were performed in triplicate in 96-well plates [see Northup et al., J. Biol. Chem., 257, 11416-23 (1982)]. To each well the following was added and allowed to incubate for 2 hours at 25° C.: 0.25 μl of a 2× buffer (5 mM HEPES pH 7.8, 1 μM GTPγS, 4 μCi/ml [³⁵S]GTPγS); 10 μl 50 mM dextromethorphan or doxylamine; and 15 μl (6.7 mg/15 ml) NCI-H7-16 cell membranes or supernatant (supernatant from NCI-H716 cells spun at 900 g (S1), pellets from NCI-H716 cells spun at 900 g (P1), and pellets from NCI-H716 cells spun at 100,000 g (P2)). Where indicated, transducin was also added (4 μg/50 μl assay). The total assay volume per well was 50 μl.

To separate the free [³⁵S] GTPγS from the [³⁵S] GTPγS that bound to G proteins, 40 μl of the mixture was filtered using a pre-wet Millipore Multiscreen Filtration opaque 96-well plate (0.45 μm mixed cellulose acetate, #MHABN4550). The samples were washed three times with 0.2 ml ice-cold wash buffer (0.5 M Tris-HCl, 0.04 M-magnesium chloride 6H₂₀, 1 M sodium chloride) using a Whatman Polyfiltronics manifold under pressure from a Gast vacuum pump (#DOA-P104 AA) set to 10 PSI. The rubber manifold of the Multiscreen plates was removed and the filters were allowed to dry under warm lights and a fan for 15 minutes.

The plates were then fitted with adapter plates (Packard #6005178) and 30 μl Microscint PS (#6013631) was added to each well. Next, the plates were heat sealed with Packard TopSeal-S sealing film (#6005161) using a Packard Micromate 496 heat sealer. Radioactivity bound to the filter plates was counted in a Packard Top Count NXT scintillation counter.

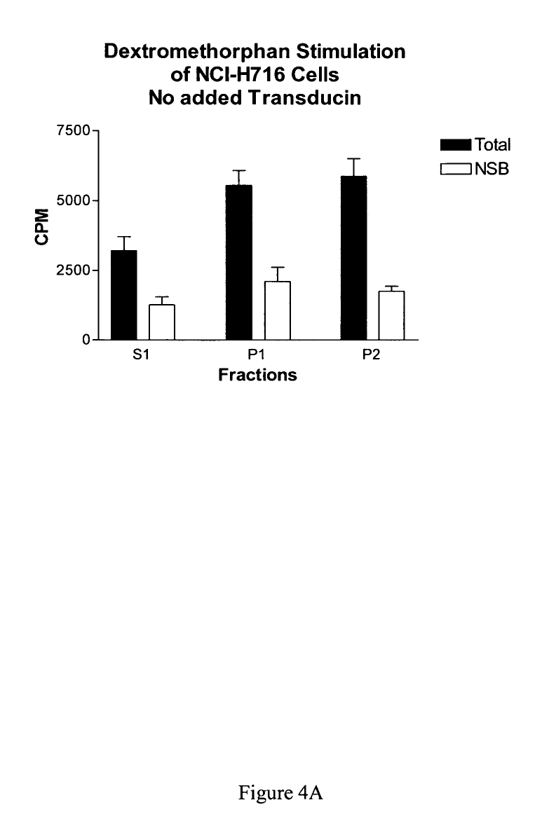

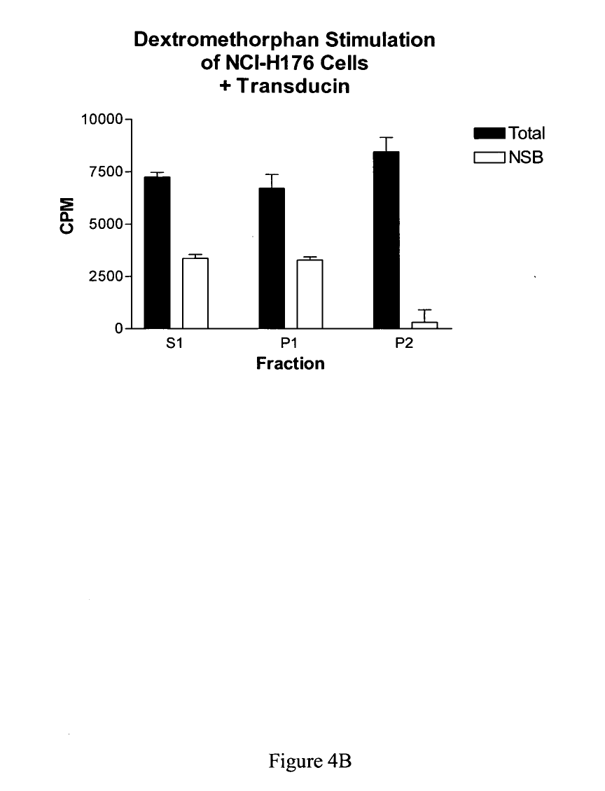

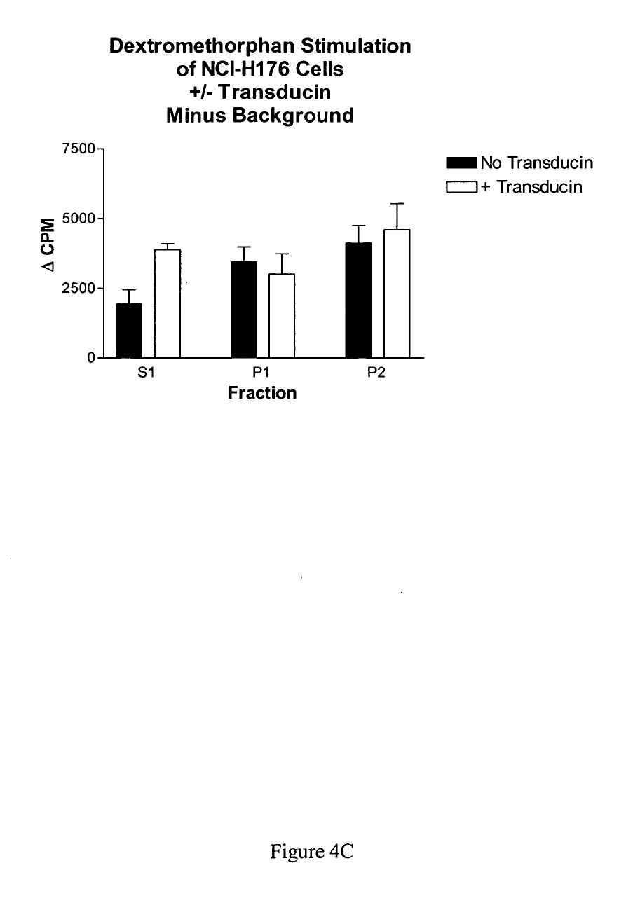

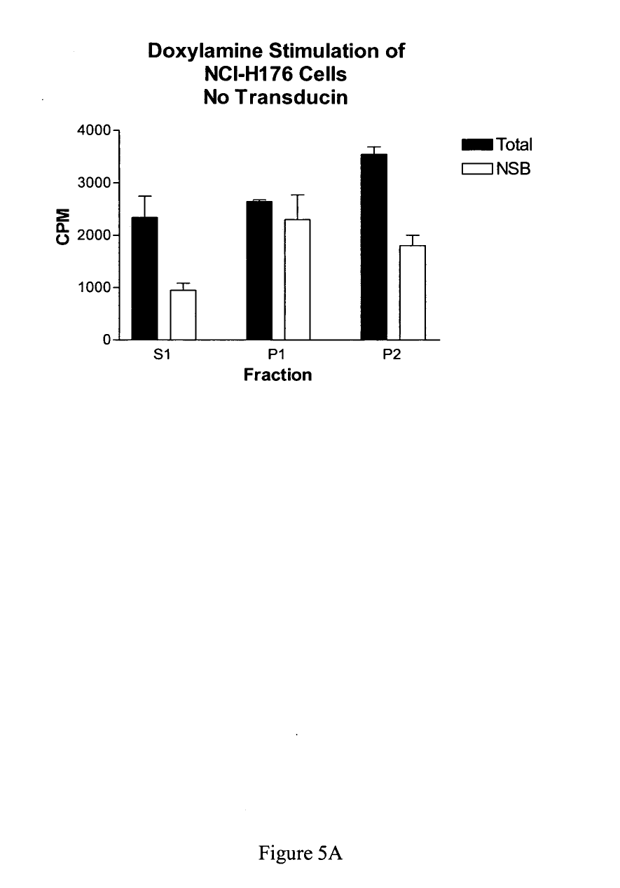

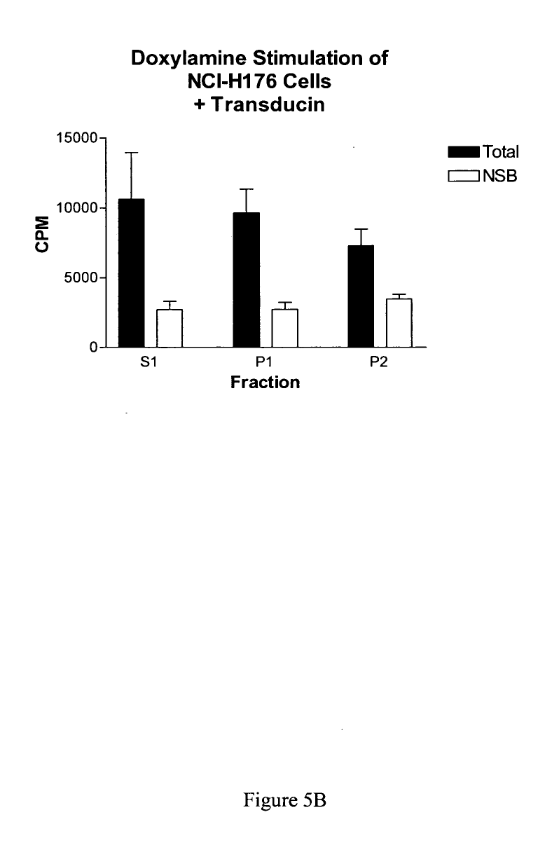

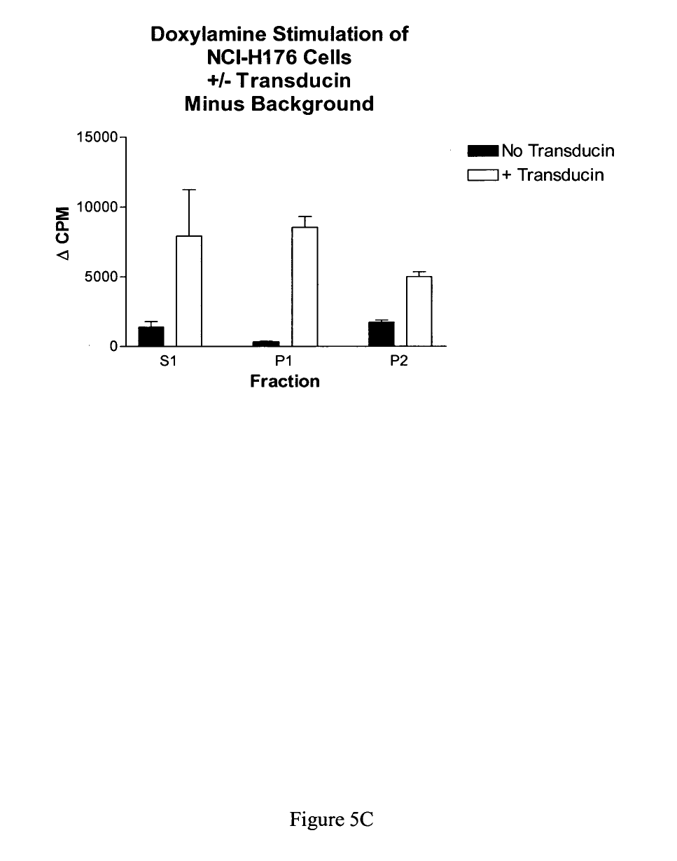

As shown in FIGS. 4 and 5, this assay measures endogenous GTPγS binding to G proteins on NCI-H716 cells. FIG. 4 demonstrates that bitter compounds, like dextromethorphan and doxylamine, increase this binding due to activation of bitter receptors and G protein signaling as compared to non-specific binding (NSB). As shown in FIG. 4A, exposure to dextromethorphan increased the binding to G proteins even in the absence of transducin, while exposure to transducin had little effect on the level of binding [see FIG. 4C]. However, for doxylamine the addition of transducin significantly increased the binding to G proteins [see FIG. 5C].

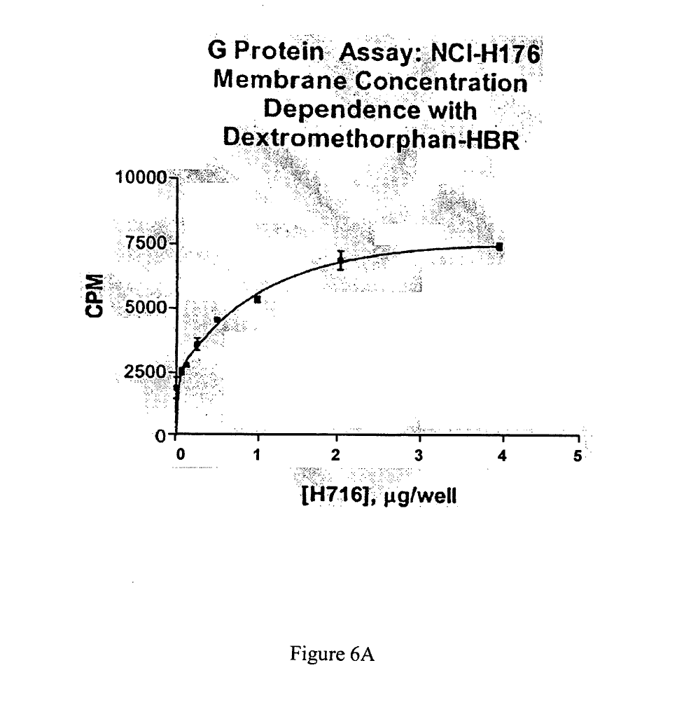

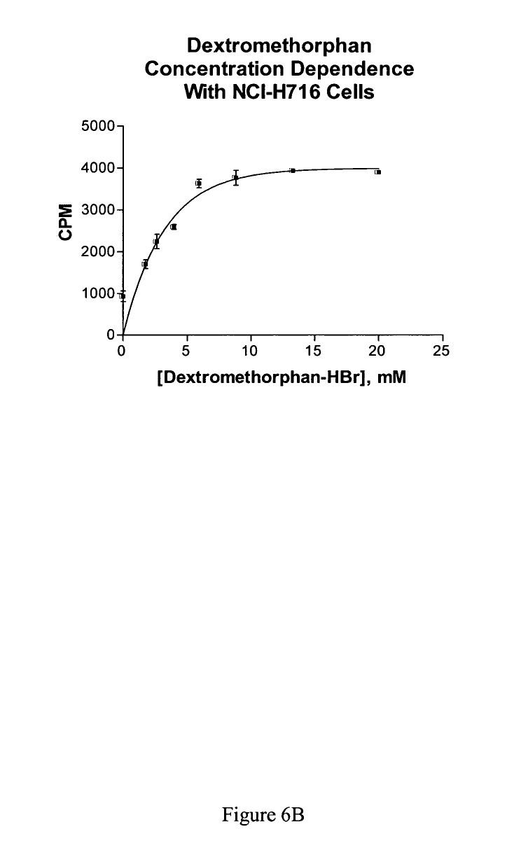

The same [³⁵S] GTPγS binding assay was used to calculate the quantity of NCI-H716 cell membranes needed to saturate 10 mM of dextromethorphan. As shown in FIG. 6A, 2 μg of NCI-H716 cell membranes were saturated by 10 mM dextromethorphan. Also assayed was the amount of dextromethorphan needed to saturate 2 μg of NCI-H716 cell membranes. As shown in FIG. 6B, 10 mM of dextromethorphan was needed to saturate 2 μg of NCI-H716 cell membranes.

As those skilled in the art will appreciate, numerous changes and modifications may be made to the preferred embodiments of the invention without departing from the spirit of the invention. It is intended that all such variations fall within the scope of the invention.

The entire disclosure of each publication cited herein is hereby incorporated by reference.

1. A method of testing whether a compound affects taste transduction comprising:
(a) contacting a human gastrointestinal cell or its membrane with the compound, wherein the cell or membrane comprises one or more taste signaling proteins; and
(b) evaluating the effect of the compound on the cell or membrane.

2. A method of identifying a modulator of taste transduction comprising:
(a) contacting a human gastrointestinal cell or its membrane with a tastant, wherein the cell or membrane comprises one or more taste signaling proteins;
(b) contacting the cell or its membrane with a compound; and
(c) evaluating the compound's effect on tastant-mediated taste transduction,
wherein a compound that alters tastant-mediated taste transduction is a modulator.

3. A method of identifying a mimic of a tastant comprising:
(a) contacting a human gastrointestinal cell or its membrane with a tastant, wherein the cell or membrane comprises one or more taste signaling proteins;
(b) evaluating the effect of the tastant on the cell or membrane;
(c) in a separate experiment, contacting the cell or its membrane with a compound;
(d) evaluating the effect of the compound on the cell or membrane; and
(e) comparing the effect of the tastant with the effect of the compound;
wherein a compound that affects the cell or membrane in the same manner as the tastant is a mimic of the tastant.

4. A method of testing whether a compound affects both taste transduction and signal transduction of one or more gastrointestinal protein hormones, neurotransmitters or soluble mediators involved in metabolism comprising:
(a) contacting a human gastrointestinal cell with the compound, wherein the cell comprises one or more taste signaling proteins and is also capable of synthesizing or secreting the one or more gastrointestinal protein hormones, neurotransmitters or soluble mediators;
(b) evaluating the effect of the compound on the one or more taste signaling proteins; and
(c) evaluating the effect of the compound on the cell's synthesis or secretion of the one or more gastrointestinal protein hormones, neurotransmitters or soluble mediators.

5. A method of testing whether a modulator of claim 2 also affects signal transduction of one or more gastrointestinal protein hormones, neurotransmitters or soluble mediators involved in metabolism comprising:
(a) contacting a human gastrointestinal cell that comprises one or more taste signaling proteins and is also capable of synthesizing or secreting the one or more gastrointestinal protein hormones, neurotransmitters or soluble mediators with the modulator; and
(b) evaluating the effect of the modulator on the cell's synthesis or secretion of the one or more gastrointestinal protein hormones, neurotransmitters or soluble mediators.

6. A method of testing whether a mimic of claim 3 also affects signal transduction of one or more gastrointestinal protein hormones, neurotransmitters or soluble mediators involved in metabolism comprising:
(a) contacting a human gastrointestinal cell that comprises one or more taste signaling proteins and is also capable of synthesizing or secreting the one or more gastrointestinal protein hormones, neurotransmitters or soluble mediators with the mimic; and
(b) evaluating the effect of the mimic on the cell's synthesis or secretion of the one or more gastrointestinal protein hormones, neurotransmitters or soluble mediators.

7. The method of claim 1 wherein the human gastrointestinal cell is derived from endocrine cells.

8. The method of claim 7 wherein the human gastrointestinal cell is derived from endocrine L-cells.

9. The method of claim 1 wherein the human gastrointestinal cell is an NCI-H716 cell (ATCC No. CCL-251).

10. The method of claim 4 wherein the one or more gastrointestinal protein hormones, neurotransmitters or soluble mediators involved in metabolism are selected from a group comprising: GLP-1, GLP-2, GIP, ghrelin, serotonin, epinephrine, norepinephrine and nitrogen oxide.

11. The method of claim 4 wherein the effect of the compound, modulator or mimic comprises an increase in the human gastrointestinal cell's synthesis or secretion of the one or more gastrointestinal protein hormones, neurotransmitters or soluble mediators.

12. The method of claim 4 wherein the effect of the compound, modulator or mimic comprises a decrease in the human gastrointestinal cell's synthesis or secretion of the one or more gastrointestinal protein hormones, neurotransmitters or soluble mediators.

13. The method of claim 1 wherein the human gastrointestinal cell comprises at least one of the following taste signaling proteins: T1R1, T1R2, T1R3, T1R4, T2R, Trpm5, PDE1A, PLCβ2, Gγ13, Gβ3, inositol trisphosphate receptor type 3, adenylyl cyclase isoform 8, gustducin a and transducin α.

14. The method of claim 2, wherein the tastant is a molecule from food or beverage, a medicament, a component of the medicament, a breakdown product of the component of the medicament, a preservative, a nutritional supplement, a medical or dental composition, an oral film, a cosmetic, a metallic salt, a composition used in pest control, soap, shampoo, toothpaste, mouthwash, mouthrinse, denture adhesive, glue on the surface of stamps or glue on the surface of envelopes.

15. The method of claim 14 wherein the component of the medicament is a vehicle for the medicament.

16. The method of claim 1 wherein the effect of the compound, tastant, modulator or mimic is on a signaling molecule.

17. The method of claim 16 wherein the signaling molecule is selected from a group comprising: cAMP, cGMP, IP₃, DAG, PDE and Ca²⁺.

18. The method of claim 1 wherein the effect of the compound, tastant, modulator or mimic is evaluated by measuring levels of ions, phosphorylation, dephosphorylation or transcription.

19. The method of claim 1 wherein the effect of the compound, tastant, modulator or mimic is evaluated by detecting changes in levels of ions, phosphorylation, dephosphorylation or transcription.

20. The method of claim 1 wherein the effect of the compound, tastant, modulator or mimic is evaluated by measuring levels of cAMP, cGMP, IP₃, DAG, PDE or Ca²⁺.

21. The method of claim 1 wherein the effect of the compound, tastant, modulator or mimic is evaluated by detecting changes in levels of cAMP, cGMP, IP₃, DAG, PDE or Ca²⁺.

22. The method of claim 1 wherein the effect of the compound, tastant, modulator or mimic is evaluated using an immunoassay or a bioassay.

23. The method of claim 22 wherein the immunoassay or bioassay detects the one or more taste signaling proteins.

24. The method of claim 22 wherein the immunoassay or bioassay detects the synthesis or secretion of the one or more gastrointestinal protein hormones, neurotransmitters or soluble mediators.
